(19) United States
(12) Patent Application Publication
(10) Pub. No.: US 2013/0298315 A1
(43) Pub. Date: Nov. 14, 2013

(54) EAR SOCK SYSTEMS

(71) Applicant: Duane Kamins, Bellaire, TX (US)

(72) Inventor: Duane Kamins, Bellaire, TX (US)

(21) Appl. No.: 13/756,030

(22) Filed: Jan. 31, 2013

Related U.S. Application Data
(60) Provisional application No. 61/644,871, filed on May 9, 2012.

(51) Int. Cl. A61F 11/14

(52) U.S. Cl. CPC A61F 11/14
USPC 2/410

(57) ABSTRACT

An ear protecting, noise blocking system for covering an ear includes at least one ear protecting member having, an adjustable outer rim to conform to the ear of a wearer; and a flexible noise attenuating member located on an interior portion of the ear protecting member. The ear protecting member encompasses a top portion and a bottom portion of the ear and is inter-changeable between a left ear and a right ear. The adjustable outer rim removably-couples the ear protecting member to the ear of the wearer. The flexible noise attenuating member is pres sable against the ear to create a seal to block out noise from entering the ear during desired quiet times. The flexible noise attenuating member is putty. The putty does not enter an ear canal of the ear, but it can be pressed against the ear to create a seal to block out the noise.

### CROSS-REFERENCE TO RELATED APPLICATION

The present application is related to and claims priority from prior provisional application Ser. No. 61/644,871, filed May 9, 2012 which application is incorporated herein by reference.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. 37 CFR 1.71(d).

### BACKGROUND OF THE INVENTION

The following includes information that may be useful in understanding the present invention(s). It is not an admission that any of the information provided herein is prior art, or material, to the presently described or claimed inventions, or that any publication or document that is specifically or implicitly referenced is prior art.

1. Field of the Invention

The present invention relates generally to the field of noise reducing devices and more specifically relates to an ear protecting, noise blocking system for blocking out noise while a person sleeps.

2. Description of the Related Art

Sleeping typically comprises a sizable portion of a 24 hour day for most individuals; most requiring about 8 hours. Sleeping in the same bed or the same room as someone who snores can be difficult. If the snorer falls asleep first, it may prevent the other person from falling asleep. The snorer can also wake up the other person in the middle of the night, and it can be difficult to fall back asleep. Additionally, many people have a difficult time falling asleep if they can hear any type of noise, not just someone who is snoring. Without a good night's sleep, people can be lethargic and can lack energy throughout their day which is undesirable.

A common solution to mask snoring is the use of earplugs. An earplug is a device that is meant to be inserted in the ear canal to protect the user's ears from loud noises or the intrusion of water, foreign bodies, dust or excessive wind. Unfortunately, earplugs can get lodged in a user's ear or cause infections from packing wax or debris down into the ear. An effective solution is necessary.

Various attempts have been made to solve the above-mentioned problems such as those found in U.S. Pat. Nos. 5,551,090; 4,702,238; 8,006,320; 4,023,642; 437,602; and 1,368,895. This art is representative of ear covering devices. None of the above inventions and patents, taken either singly or in combination, is seen to describe the invention as claimed.

Ideally, an ear protecting, noise blocking system, should be user-friendly and safe in-use and, yet would operate reliably and be manufactured at a modest expense. Thus, a need exists for a reliable ear protecting, noise blocking system for blocking out noise while a person sleeps and to avoid the above-mentioned problems.

### BRIEF SUMMARY OF THE INVENTION

In view of the foregoing disadvantages inherent in the known noise reduction devices art, the present invention provides a novel ear protecting, noise blocking system. The general purpose of the present invention, which will be described subsequently in greater detail is to provide an ear protecting, noise blocking system for effectively blocking out noise while a person sleeps such that the user can enjoy a restful sleep.

An ear protecting, noise blocking system for covering an ear is disclosed herein comprising: at least one ear protecting member having an adjustable outer rim to (adjustably) conform to the ear of a wearer; and a flexible noise attenuating member. The ear protecting member encompasses a top portion and a bottom portion of the ear when used. The ear protecting member comprises cloth material in preferred embodiments. The ear protecting member may comprise indicia allowing customization by the wearer. The ear protecting member is disposable in certain embodiments and is inter-changeable between a left ear and a right ear to minimize manufacturing costs. The adjustable outer rim removably-couples the ear protecting member to the ear of the wearer. Further, the adjustable outer rim preferably comprises elastic. The elastic permits the ear protecting member to expand and contract to encompass the ear but remain snug at the bottom portion of the ear regardless of the size of the ear.

The flexible noise attenuating member is located on an interior portion of the ear protecting member. The flexible noise attenuating member is located in a center region of the interior portion of the ear protecting member. The flexible noise attenuating member is pressable against the ear to create a seal to block out noise from entering the ear during desired quiet times. Additionally, the flexible noise attenuating member comprises putty. The putty is preferably nontoxic and hypoallergenic. The putty does not enter an ear canal of the ear, but it can be pressed against the ear to create a seal to block out the noise.

A kit is also embodied herein for the ear protecting member in various colors and designs; with a set of user instructions.

A method of use for a ear protecting, noise blocking system comprises the steps of: locating at least one ear protecting member; grasping the ear protecting member; applying a first ear protecting member over a left ear for canceling out noise; applying a second ear protecting member over a right ear for canceling out noise; removing the first ear protecting member from the left ear when noise has subsided; removing the second ear protecting member from the right ear when noise has subsided; and discarding the first and second ear protecting members from the left ear and the right ear (in disposable embodiments).

The present invention holds significant improvements and serves as an ear protecting, noise blocking system. For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and method(s) of use for the present invention, ear protecting, noise blocking system, constructed and operative according to the teachings of the present invention.

FIG. 1 shows a perspective view illustrating an ear protecting, noise blocking system in an in-use condition according to an embodiment of the present invention.

FIG. 2 is a perspective view illustrating the ear protecting, noise blocking system in another in-use condition according to an embodiment of the present invention of FIG. 1.

FIG. 3A is a side perspective view illustrating a front portion at least one ear protecting member of the ear protecting, noise blocking system according to an embodiment of the present invention of FIGS. 1 and 2.

FIG. 3B is another side perspective view illustrating a back portion at least one ear protecting member of the ear protecting, noise blocking system according to an embodiment of the present invention of FIGS. 1 and 2.

FIG. 4 is a perspective view illustrating putty located on an interior portion of the ear protecting member of the ear protecting, noise blocking system according to an embodiment of the present invention of FIGS. 1 and 2.

FIG. 5 is a perspective view illustrating another view of the putty located on an interior portion of the at least one ear protecting member of the ear protecting, noise blocking system according to an embodiment of the present invention of FIGS. 1 and 2.

FIG. 6 is a flowchart illustrating a method of use for the ear protecting, noise blocking system according to an embodiment of the present invention of FIGS. 1-5.

The various embodiments of the present invention will hereinafter be described in conjunction with the appended drawings, wherein like designations denote like elements.

### DETAILED DESCRIPTION

As discussed above, embodiments of the present invention relate to noise canceling devices and more specifically relates to an ear protecting, noise blocking system for blocking out noise while a person sleeps and/or rests.

Generally speaking, the Ear Sock (ear protecting, noise blocking system) can act as a sound barrier, blocking out noise while a person sleeps. This unique product can comprise an elastic-gathered ear covering. The elasticity can enable the covering to expand and contract to fit over the ear easily but remain snug at the base of the ear for comfort. The covers can have nontoxic, hypoallergenic putty on the inside of the coverings. The putty cannot enter the ear canal, but it can be pressed against the ear to create a seal to block out sounds. The outside of the covering can be made from a soft cloth material that can also act as a sound barrier. Before going to bed or before trying to fall asleep, people can simply place a covering over each ear and enjoy a restful sleep.

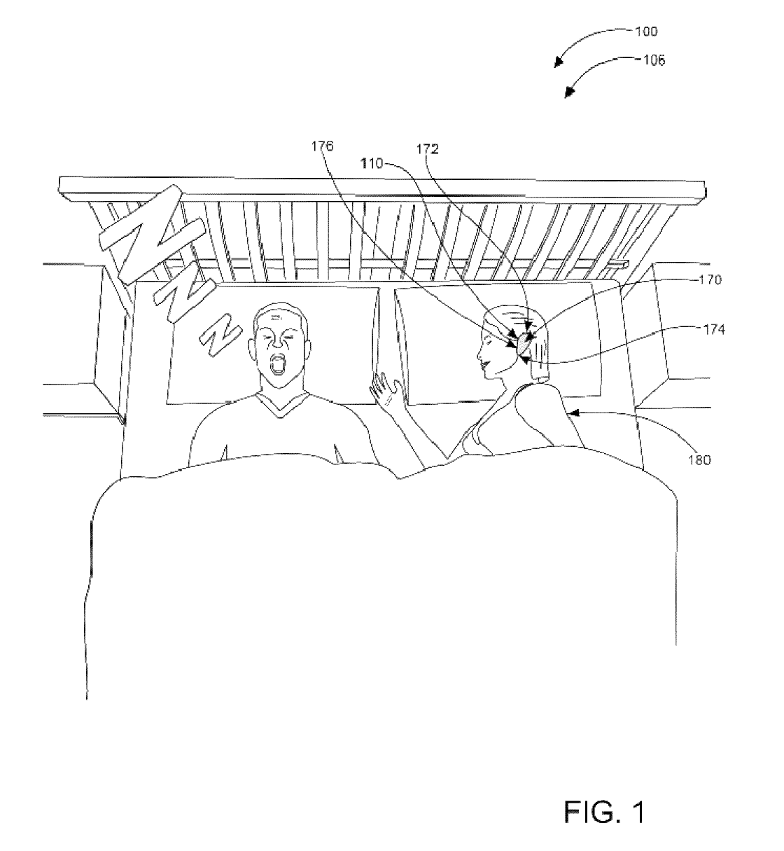

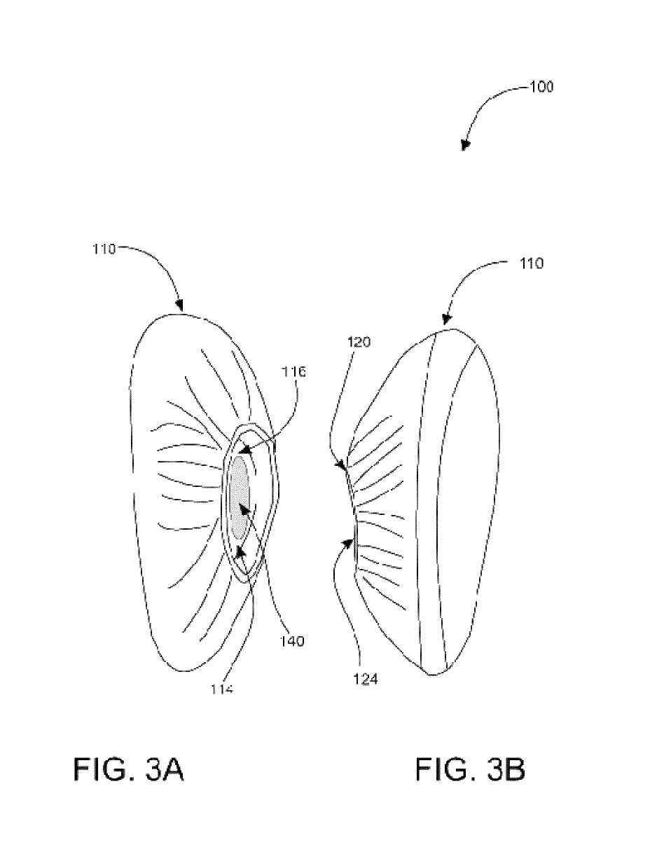

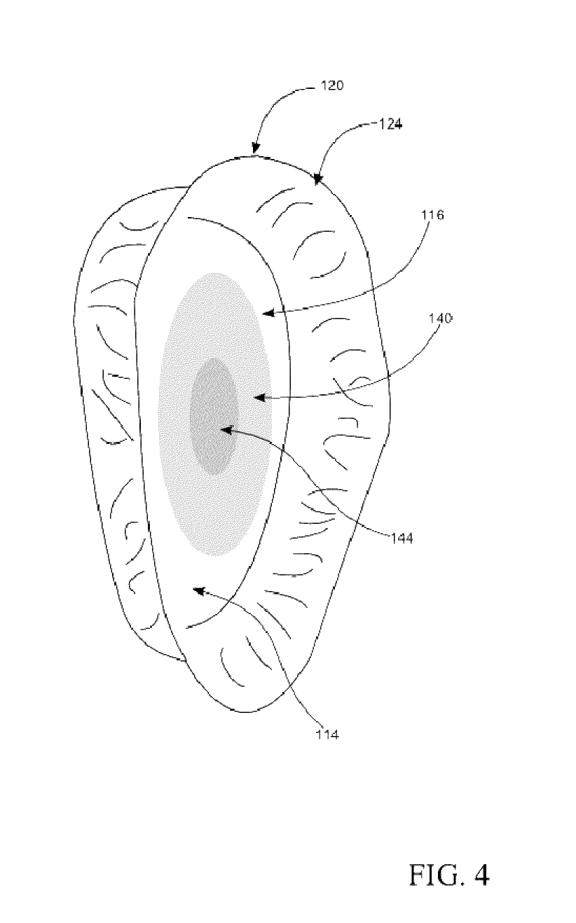

Referring now to the drawings by numerals of reference there is shown in FIGS. 1-5, various perspective views illustrating ear protecting, noise blocking system 100 according to an embodiment of the present invention.

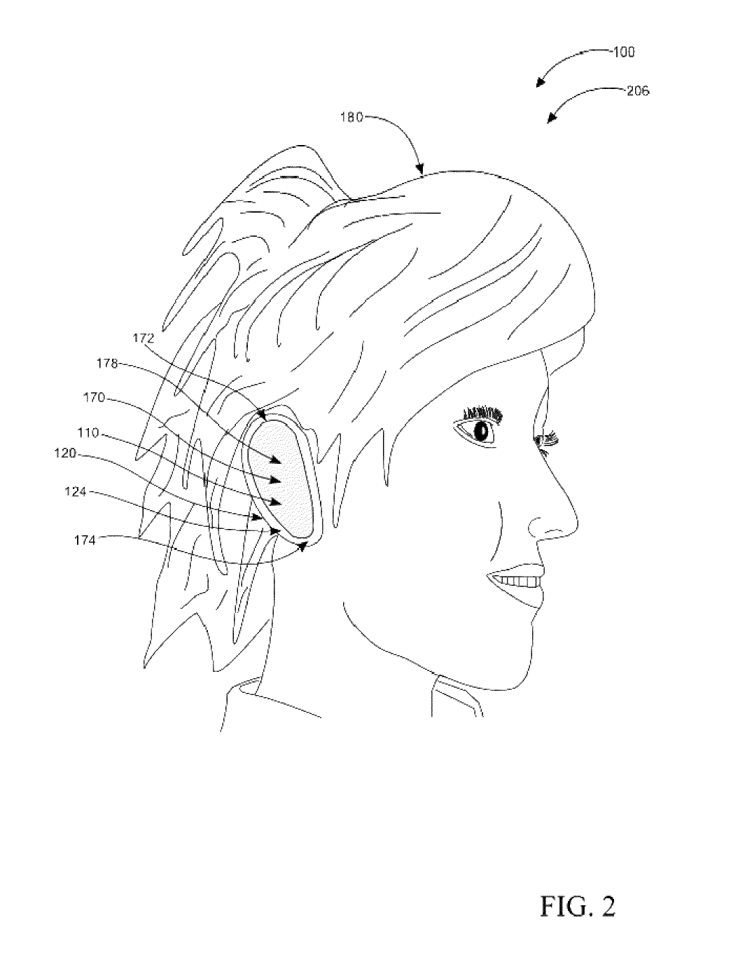

Ear protecting, noise blocking system 100 for covering ear 170 is disclosed herein comprising: at least one ear protecting member 110 having an adjustable outer rim 120 to conform to ear 170 of wearer 180; and flexible noise attenuating member 140. Ear protecting member 110 encompasses top portion 172 and bottom portion 174 of ear 170 as shown in in-use conditions 106 of FIG. 1 and in-use condition 206 of FIG. 2.

Ear protecting member 110 preferably comprises cloth material (fabric). Upon reading this specification, it should be appreciated that, under appropriate circumstances, considering such issues as user preferences, design preference, structural requirements, marketing preferences, cost, available materials, technological advances, etc., other materials such as, for example, polyethylene material, leather, or other such material, etc., may be sufficient.

Ear protecting member 110 may comprise indicia allowing customization by wearer 180. Ear protecting member 110 may also comprise at least one logo. Logo may include but is not limited to a favorite sports team or a popular brand to name a few.

Ear protecting member 110 may either be manufactured in a disposable (as previously mentioned) or in a re-useable form. Further, ear protecting member 110 is inter-changeable between left ear 176 and right ear 178. Adjustable outer rim 120 preferably removably-couples ear protecting member 110 to ear 170 of wearer 180. Further, adjustable outer rim 120 preferably comprises elastic 124. Elastic 124 permits ear protecting member 110 to expand and contract to encompass ear 170 but remain snug at bottom portion 174 of ear 170. It should be noted adjustable outer rim 120 may comprises a user-adjustable cord (not shown) in alternate embodiments.

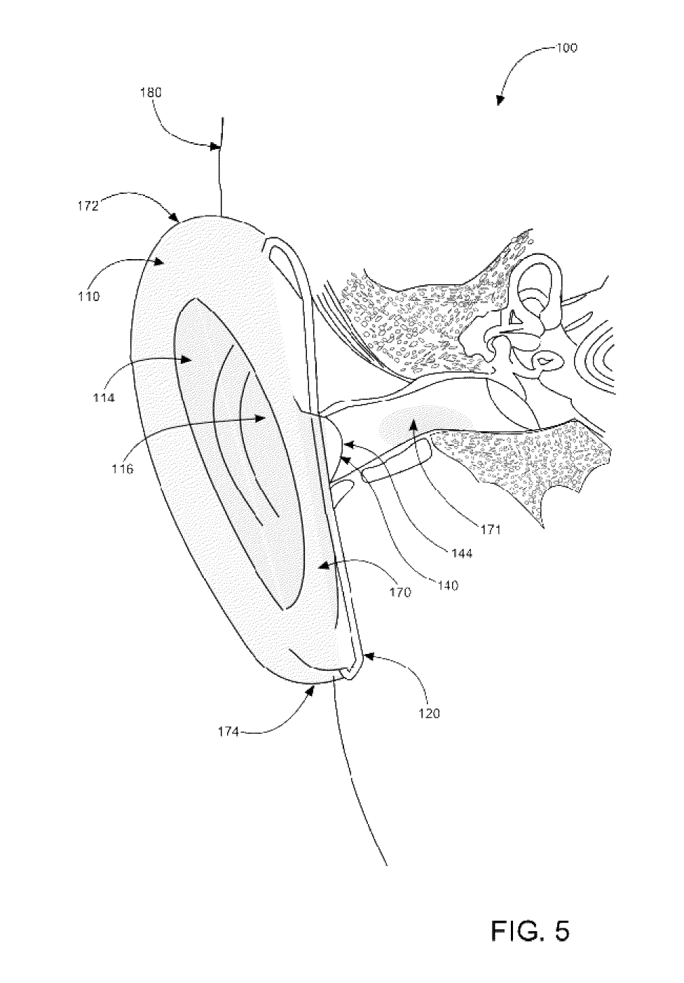

Flexible noise attenuating member 140 is located in center region 116 on interior portion 114 of ear protecting member 110. Flexible noise attenuating member 140 is pressable against ear 170 to create a seal to block out noise from entering ear 170 during desired quiet times as shown best in FIG. 5. Additionally, flexible noise attenuating member 140 preferably comprises putty 144. Putty 144 is nontoxic and hypoallergenic in preferred embodiments. Putty 144 does not enter ear canal 171 of ear 170, but it can be pressed against ear 170 to create a seal to block out the noise. This is designed in this manner to avoid pushing unwanted debris into ear canal 171 of ear 170.

Ear protecting, noise blocking system 100 according to an embodiment of the present invention of FIGS. 1-4 may comprise kit 450. Kit 450 may comprise the following parts: a plurality of ear protecting members 110 in various colors and designs; and a set of user instructions. Upon reading this specification, it should be appreciated that, under appropriate circumstances, considering such issues as design preference, user preferences, marketing preferences, cost, structural requirements, available materials, technological advances, etc., other kit contents or arrangements such as, for example, including more or less components, customized parts, different material combinations, parts may be sold separately such as adhesives, etc., may be sufficient.

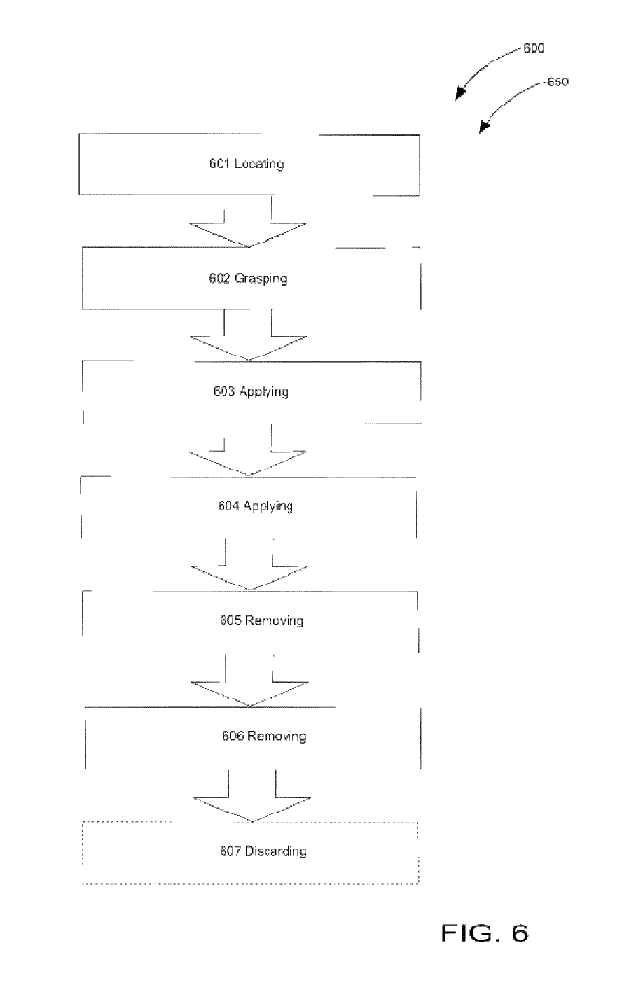

Referring now to FIG. 6, flowchart 650 illustrating method of use 600 for ear protecting, noise blocking system 100 according to an embodiment of the present invention of FIGS. 1-5.

Method of use 600 for ear protecting, noise blocking system 100 preferably comprises the steps of: step one 601 locating at least one ear protecting member 110; step two 602 grasping ear protecting member 110; step three 603 applying a first ear protecting member over left ear 176 for canceling out noise; step four 604 applying a second ear protecting member over right ear 178 for canceling out noise; step five 605 removing the first ear protecting member from left ear 176 when noise has subsided; step six 606 removing the second ear protecting member from right ear 178 when noise has subsided; and step seven 607 discarding the first and second ear protecting members from left ear 176 and right ear 178. Steps may include adjusting once donned.

It should be noted that step 607 is an optional step and may not be implemented in all cases as with non-disposable versions. Optional steps of method 500 are illustrated using dotted lines in FIG. 5 so as to distinguish them from the other steps of method 500.

It should be noted that the steps described in the method of use can be carried out in many different orders according to user preference. The use of “step of” should not be interpreted as “step for”, in the claims herein and is not intended to invoke the provisions of 35 U.S.C. §112, ¶6. Upon reading this specification, it should be appreciated that, under appropriate circumstances, considering such issues as design preference, user preferences, marketing preferences, cost, structural requirements, available materials, technological advances, etc., other methods of use arrangements such as, for example, different orders within above-mentioned list, elimination or addition of certain steps, including or excluding certain maintenance steps, etc., may be sufficient.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. An ear protecting, noise blocking system for covering an ear comprising:
at least one ear protecting member having,
an adjustable outer rim to conform to said ear of a wearer; and
a flexible noise attenuating member;
wherein said ear protecting member encompasses a top portion and a bottom portion of said ear when worn;
wherein said adjustable outer rim removably-couples said ear protecting member to said ear of said wearer;
wherein said flexible noise attenuating member is located on an interior portion of said ear protecting member; and
wherein said flexible noise attenuating member is pres sable against said ear to create a seal to block out noise from entering said ear during desired quiet times.

2. The ear protecting, noise blocking system of claim 1 wherein said adjustable outer rim comprises elastic.

3. The ear protecting, noise blocking system of claim 1 wherein said adjustable outer rim comprises a user-adjustable cord.

4. The ear protecting, noise blocking system of claim 2 wherein said elastic permits said ear protecting member to expand and contract to encompass said ear but yet remain snug at said bottom portion of said ear.

5. The ear protecting, noise blocking system of claim 1 wherein said ear protecting member comprises cloth material.

6. The ear protecting, noise blocking system of claim 1 wherein said ear protecting member comprises polyethylene material.

7. The ear protecting, noise blocking system of claim 1 wherein said flexible noise attenuating member comprises putty.

8. The ear protecting, noise blocking system of claim 7 wherein said putty is nontoxic.

9. The ear protecting, noise blocking system of claim 7 wherein said putty is hypoallergenic.

10. The ear protecting, noise blocking system of claim 7 wherein said putty does not enter an ear canal of said ear, but it can be pressed against said ear to create a seal to block out said noise.

11. The ear protecting, noise blocking system of claim 7 wherein said flexible noise attenuating member is located in a center region of said interior portion of said ear protecting member.

12. The ear protecting, noise blocking system of claim 1 wherein said ear protecting member comprises at least one logo.

13. The ear protecting, noise blocking system of claim 1 wherein said ear protecting member comprises indicia allowing customization by said wearer.

14. The ear protecting, noise blocking system of claim 1 wherein said ear protecting member is disposable.

15. The ear protecting, noise blocking system of claim 1 wherein said ear protecting member is re-useable.

16. The ear protecting, noise blocking system of claim 1 wherein said ear protecting member is inter-changeable between a left said ear and a right said ear.

17. An ear protecting, noise blocking system for covering an ear comprising:
at least one ear protecting member having,
an adjustable outer rim to conform to said ear of a wearer; and
a flexible noise attenuating member;
wherein said ear protecting member encompasses a top portion and a bottom portion of said ear;
wherein said ear protecting member comprises cloth material;
wherein said ear protecting member comprises indicia allowing customization by said wearer;
wherein said ear protecting member is disposable;
wherein said ear protecting member is inter-changeable between a left said ear and a right said ear;
wherein said adjustable outer rim removably-couples said ear protecting member to said ear of said wearer;
wherein said adjustable outer rim comprises elastic;
wherein said elastic permits said ear protecting member to expand and contract to encompass said ear but remain snug at said bottom portion of said ear;
wherein said flexible noise attenuating member is located on an interior portion of said ear protecting member;
wherein said flexible noise attenuating member is located in a center region of said interior portion of said ear protecting member;
wherein said flexible noise attenuating member is pres sable against said ear to create a seal to block out noise from entering said ear during desired quiet times;
wherein said flexible noise attenuating member comprises putty;
wherein said putty is nontoxic and hypoallergenic; and
wherein said putty does not enter an ear canal of said ear, but it can be pressed against said ear to create a seal to block out said noise to provide a restful sleep.

18. The ear protecting, noise blocking system of claim 17 further comprising a kit including:
said ear protecting member(s) in various colors and designs; and a set of user instructions.

19. A method of using an ear protecting, noise blocking system comprising the steps of:
locating at least one ear protecting member;
grasping said ear protecting member;
applying a first said ear protecting member over a left ear for canceling out noise;
applying a second said ear protecting member over a right ear for canceling out said noise;
removing said first said ear protecting member from said left ear when said noise has subsided; and
removing said second said ear protecting member from said right ear when said noise has subsided.

20. The method of using the ear protecting, noise blocking system of claim 19 further comprising the step of discarding said first and said second ear protecting members from said left ear and said right ear.
